(19) United States
(12) Patent Application Publication
(10) Pub. No.: US 2025/0038681 A1
(43) Pub. Date: Jan. 30, 2025

(54) METHODS OF DYNAMICALLY STOPPING A MOTOR

(71) Applicant: Auris Health, Inc., Redwood City, CA (US)

(72) Inventors: Shane Daniel KENT, Millbrae, CA (US); Nicholas J. EYRE, Redwood City, CA (US); Sven WEHRMANN, Redwood City, CA (US)

(21) Appl. No.: 18/893,862

(22) Filed: Sep. 23, 2024

Related U.S. Application Data
(63) Continuation of application No. PCT/IB2023/052589, filed on Mar. 16, 2023.
(60) Provisional application No. 63/323,468, filed on Mar. 24, 2022.

(51) Int. Cl. H02P 3/22; A61B 34/30; A61B 34/37; B25J 9/12

(52) U.S. Cl. CPC H02P 3/22; A61B 34/37; B25J 9/12; A61B 2034/302; A61B 2034/303

(57) ABSTRACT

A medical system may include a motor of a robotic component, a driver circuit coupled to the motor, and a shunting circuit coupled to the driver circuit. The shunting circuit may be configured to shunt the driver circuit for a set amount of time to stop the motor. The shunting circuit may include a capacitor (C) and a first resistor (R), the capacitor and first resistor having an RC time constant, where the set amount of time corresponds to the RC time constant. Methods for operating a shunting circuit are also disclosed herein.

### PRIORITY

This application is a continuation of International Patent Application No. PCT/IB2023/052589, filed Mar. 16, 2023, entitled “Methods of Dynamically Stopping a Motor,” which claims priority to U.S. Provisional Patent Application No. 63/323,468, entitled “Methods of Dynamically Stopping a Motor,” filed Mar. 24, 2022, the disclosures of each of which are incorporated by reference herein, in their entirety.

### TECHNICAL FIELD

The systems and methods disclosed herein are directed to dynamically stopping motors, and more particularly to using shunting techniques to dynamically stop motors.

### BACKGROUND

A robotically enabled medical system is capable of performing a variety of medical procedures, including both minimally invasive procedures, such as laparoscopy, and non-invasive procedures, such as endoscopy (e.g., bronchoscopy, ureteroscopy, gastroscopy, etc.). Such robotic medical systems can include one or more robotic arms configured to control the movement of medical tool(s) during a given medical procedure. Each robotic arm can include one or more motors to drive its joints. In an emergency stop scenario, the motor(s) need to be stopped quickly and safely. In some circumstances, the motor(s) may be back-drivable after stopping so that an operator can manually move the robotic arm away from the patient or out of the way.

### SUMMARY

The present disclosure describes methods and circuits to stop a motor quickly using hardware control rather than firmware or software control. Hardware control for stopping a motor can be beneficial in a fault or emergency stop situation, as it enables the motor to stop quickly even if a fault is present in one of the sensors or components normally used for robot control. Stopping motors on a medical system quickly can be important in ensuring that a robot arm and an associated medical tool do not travel significantly after a fault is detected, which in turn enhances patient safety.

When robot joints are stopped under closed-loop control (e.g., in a non-fault scenario), the kinetic energy of the joints is converted by the motors into electrical energy which passes back onto the voltage bus. The electrical energy passing back onto the voltage bus can raise the voltage of the bus unless resistors are included that can convert some of the energy into heat to prevent large voltage spikes. A closed-loop control can manage the current passing through the motors, and thus the closed-loop control can remove (or convert) the kinetic energy quickly.

In an emergency stop scenario involving robot arm joints, kinetic energy can be removed from the joints and converted into mechanical energy (e.g., associated with friction caused by brakes) and/or thermal energy (e.g., heat generated by a resistor). However, in some configurations, the brakes in the robot arms are sized primarily for static holding, and therefore may not provide sufficient friction to stop the joints quickly. Furthermore, converting all the kinetic energy into friction can wear the brakes prematurely.

Shunting the motor provides a way to transfer some of the energy out of the motor and convert into heat using a resistor, e.g., shunting the individual phases (e.g., by controlling the transistors) or by shunting the whole bus. In some robotic systems the motors have low internal resistance, therefore if the motors are shunted without additional external resistance, the current can spike. The current spike can be limited or reduced by adding additional resistance to the shunting path.

Accordingly, in one aspect, a medical system includes a motor of a robotic component, a driver circuit coupled to the motor, and a shunting circuit coupled to the driver circuit and configured to shunt the driver circuit for a set amount of time to stop the motor, the shunting circuit comprising a capacitor (C) and a first resistor (R), the capacitor and first resistor having an RC time constant, where the set amount of time corresponds to the RC time constant.

In another aspect, a medical system includes a motor of a robotic component, a driver circuit coupled to the motor, and a shunting circuit coupled to the driver circuit and configured to shunt the driver circuit to stop the motor. The shunting circuit includes a first switch coupling the driver circuit to a voltage source, a second switch coupling the driver circuit to an electrical ground, a current sensor coupled to the driver circuit and configured to measure a current in the driver circuit, and a control component coupled to the current sensor, the first switch, and the second switch. The control component is configured to, in response to a fault signal, open the first switch, and selectively open and close the second switch to maintain the current in the driver circuit within a set range while stopping the motor.

In another aspect, a medical system includes a motor of a robotic component, a driver circuit coupled to the motor, and a shunting circuit coupled to the driver circuit. The shunting circuit includes a switch electrically coupled between a high voltage line of the driver circuit and an electrical ground, a resistor electrically coupled between the high voltage line of the driver circuit and an electrical ground, and a capacitor coupled to the switch for shunting the driver circuit over a set amount of time.

In another aspect, a method for stopping a motor of a robotic component includes driving the motor via a driver circuit, and, while the motor is being driven, receiving a fault signal. In response to the fault signal, the driver circuit is shunted to stop the motor. The shunting includes decoupling a driver circuit for the motor from a voltage source, and coupling the driver circuit to an electrical ground via a resistor, where the driver circuit is decoupled from the electrical ground in accordance with charge draining from a capacitor. In some embodiments, the method is performed by one or more processors executing instructions store in memory.

In yet another aspect, a method for stopping a motor of a robotic component includes driving the motor via a driver circuit, and, while the motor is being driven, receiving a fault signal. In response to the fault signal, the driver circuit is shunted to stop the motor. The shunting includes decoupling a driver circuit for the motor from a voltage source, and selectively coupling the driver circuit to an electrical ground until the motor stops. The selective coupling including monitoring a current in the driver circuit, in accordance with the current exceeding a first predetermined threshold, decoupling the driver circuit from the electrical ground, and, in accordance with the current going below a second predetermined threshold, recoupling the driver circuit to the electrical ground. In some embodiments, the method is performed by one or more processors executing instructions store in memory.

The systems, methods, and devices of this disclosure each have several innovative aspects, no single one of which is solely responsible for the desirable attributes disclosed herein.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s), according to some embodiments.

FIG. 2 depicts further aspects of the robotic system of FIG. 1, according to some embodiments.

FIG. 3 illustrates an embodiment of the robotic system of FIG. 1 arranged for ureteroscopy, according to some embodiments.

FIG. 4 illustrates an embodiment of the robotic system of FIG. 1 arranged for a vascular procedure, according to some embodiments.

FIG. 5 illustrates an embodiment of a table-based robotic system arranged for a bronchoscopy procedure, according to some embodiments.

FIG. 6 illustrates an example system configured to stow robotic arm(s), according to some embodiments.

FIG. 7 illustrates an alternative embodiment of a table-based robotic system, according to some embodiments.

FIG. 8 illustrates an end view of the table-based robotic system of FIG. 7, according to some embodiments.

FIG. 9 illustrates an end view of a table-based robotic system with robotic arms attached thereto, according to some embodiments.

FIG. 10 illustrates an example instrument driver, according to some embodiments.

FIG. 11 illustrates an example medical instrument with a paired instrument driver, according to some embodiments.

FIG. 12 illustrates an alternative design for an instrument driver and instrument where the axes of the drive units are parallel to the axis of the elongated shaft of the instrument, according to some embodiments.

FIG. 13 illustrates an instrument having an instrument-based insertion architecture, according to some embodiments.

FIG. 14 illustrates an example controller, according to some embodiments.

FIG. 15A illustrates an example motor driver circuit for a motor, according to some embodiments.

FIGS. 15B-15C illustrate example shunting paths for the example motor driver circuit of FIG. 15A, according to some embodiments.

FIG. 16A illustrates an example shunting circuit for a motor, according to some embodiments.

FIGS. 16B-16D illustrate example operating states for the shunting circuit of FIG. 16A, according to some embodiments.

FIGS. 17A-17B illustrate example shunting characteristics for the shunting circuit of FIG. 16A, according to some embodiments.

FIG. 18 illustrates another example shunting circuit for a motor, according to some embodiments.

FIGS. 19A-19D illustrate example shunting characteristics for the shunting circuit of FIG. 18, according to some embodiments.

FIGS. 20A-20B are a flowchart illustrating an example method for shunting a motor in accordance with some embodiments.

FIG. 21 is a schematic diagram illustrating electronic components of a medical system in accordance with some embodiments.

### DETAILED DESCRIPTION

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

In addition to performing the breadth of procedures, the system may provide additional benefits, such as enhanced imaging and guidance to assist the physician. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations.

### A. Robotic System—Cart.

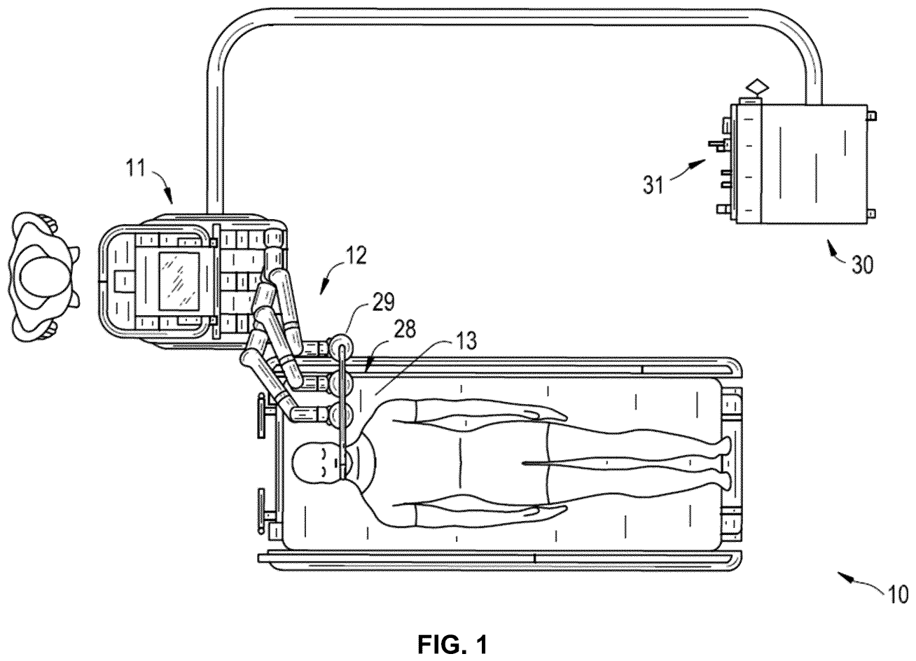

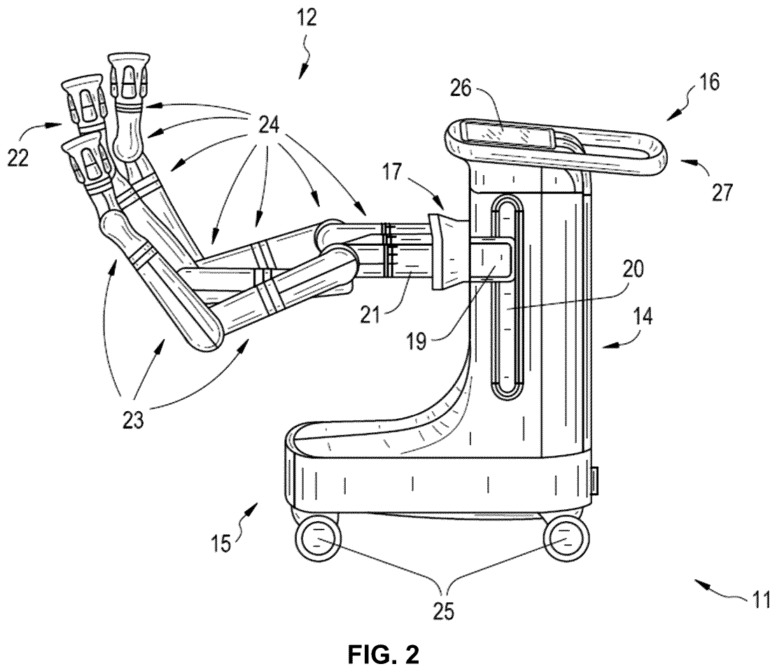

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope. FIG. 2 depicts an example embodiment of the cart in greater detail.

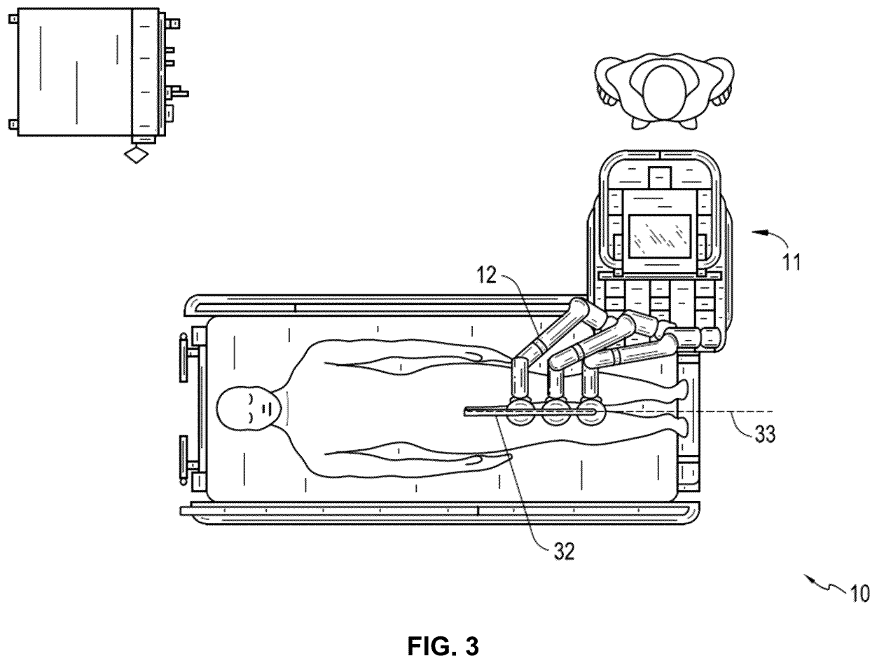

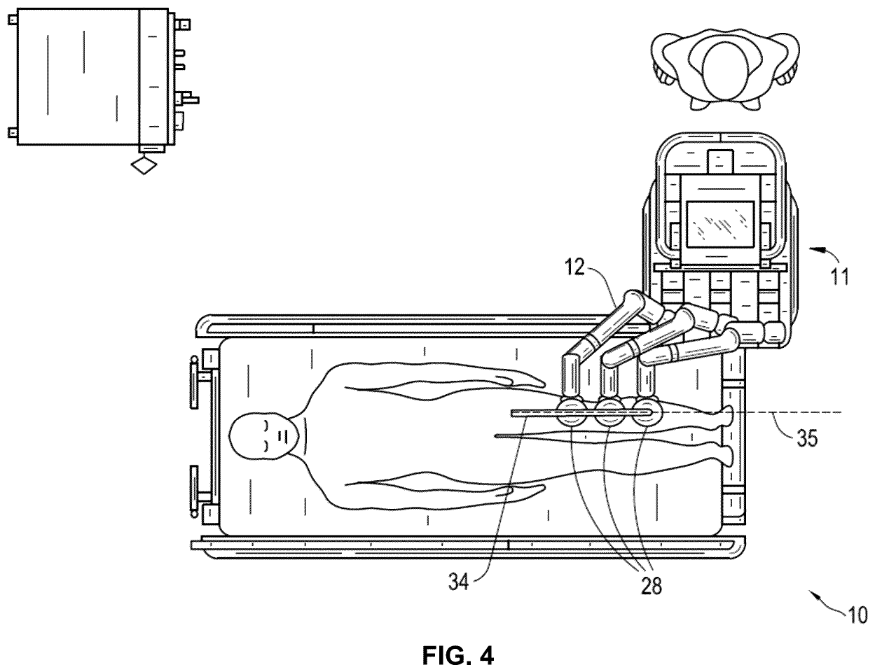

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines (such as in FIGS. 3 and 4), and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to respect the potentially cancerous tissue. In some instances, diagnostic and therapeutic treatments can be delivered in separate procedures. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of the tower 30. In some embodiments, irrigation and aspiration capabilities may be delivered directly to the endoscope 13 through separate cable(s).

The tower 30 may also include support equipment for the sensors deployed throughout the robotic system 10. For example, the tower 30 may include optoelectronics equipment for detecting, receiving, and processing data received from the optical sensors or cameras throughout the robotic system 10. In combination with the control system, such optoelectronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in the system 10 are generally designed to provide both robotic controls as well as preoperative and real-time information of the procedure, such as navigational and localization information of the endoscope 13. When the console 31 is not the only console available to the physician, it may be used by a second operator, such as a nurse, to monitor the health or vitals of the patient and the operation of the system 10, as well as to provide procedure-specific data, such as navigational and localization information. In other embodiments, the console 31 is housed in a body that is separate from the tower 30.

FIG. 2 provides a detailed illustration of an embodiment of the cart 11 from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage 17 at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of the robotic arms 12 to be angled in a variety of configurations.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm 12. Each of the robotic arms 12 may have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and robotic arms 12 over the floor. Accordingly, the cart base 15 houses heavier components, such as electronics, motors, power supply, as well as components that either enable movement and/or immobilize the cart 11. For example, the cart base 15 includes rollable wheel-shaped casters 25 that allow for the cart 11 to easily move around the room prior to a procedure. After reaching the appropriate position, the casters 25 may be immobilized using wheel locks to hold the cart 11 in place during the procedure.

Positioned at the vertical end of the column 14, the console 16 allows for both a user interface for receiving user input and a display screen (or a dual-purpose device such as, for example, a touchscreen 26) to provide the physician user with both preoperative and intra-operative data. Potential preoperative data on the touchscreen 26 may include preoperative plans, navigation and mapping data derived from preoperative computerized tomography (CT) scans, and/or notes from preoperative patient interviews. Intra-operative data on display may include optical information provided from the tool, sensor and coordinate information from sensors, as well as vital patient statistics, such as respiration, heart rate, and/or pulse.

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

FIG. 4 illustrates an embodiment of a robotically-enabled system 10 similarly arranged for a vascular procedure. In a vascular procedure, the system 10 may be configured such that the cart 11 may deliver a medical instrument 34, such as a steerable catheter, to an access point in the femoral artery in the patient's leg. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

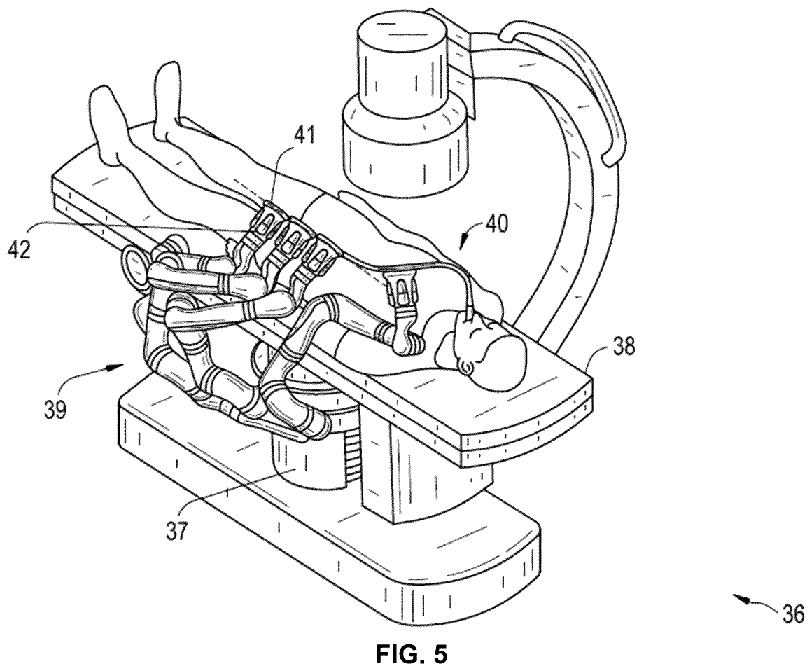

Embodiments of the robotically-enabled medical system may also incorporate the patient's table. Incorporation of the table may reduce the amount of capital equipment within the operating room by removing the cart, which allows greater access to the patient. FIG. 5 illustrates an embodiment of such a robotically-enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around the table 38.

As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient.

The system 36 may also include a tower (not shown) that divides the functionality of the system 36 between the table and the tower to reduce the form factor and bulk of the table. As in earlier disclosed embodiments, the tower may provide a variety of support functionalities to the table, such as processing, computing, and control capabilities, power, fluidics, and/or optical and sensor processing. The tower may also be movable to be positioned away from the patient to improve physician access and de-clutter the operating room. The tower may also include a master controller or console that provides both a user interface for user input, such as keyboard and/or pendant, as well as a display screen (or touchscreen) for preoperative and intra-operative information, such as real-time imaging, navigation, and tracking information. In some embodiments, the tower may also contain holders for gas tanks to be used for insufflation.

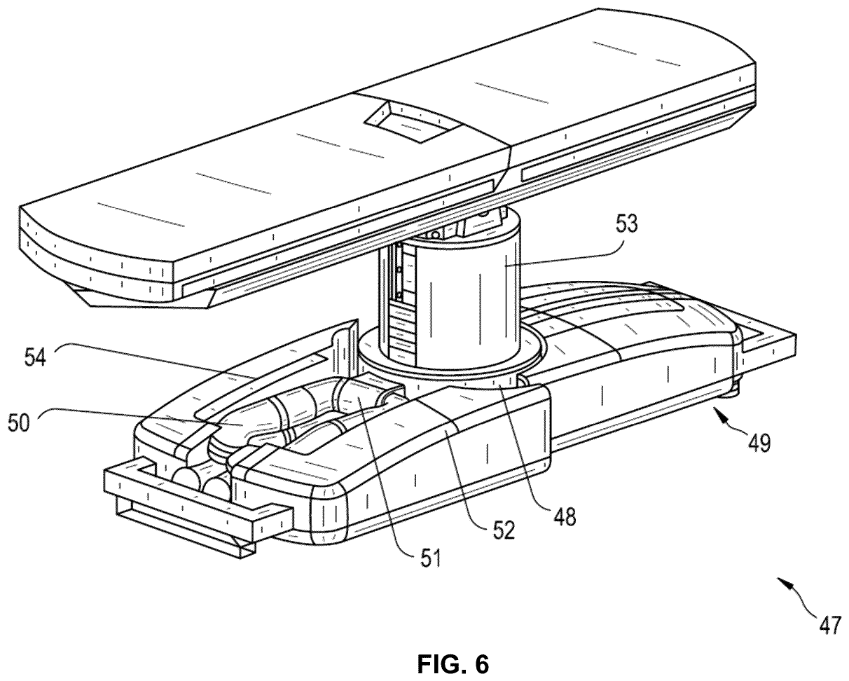

In some embodiments, a table base may stow and store the robotic arms when not in use. FIG. 6 illustrates a system 47 that stows robotic arms in an embodiment of the table-based system. In the system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use.

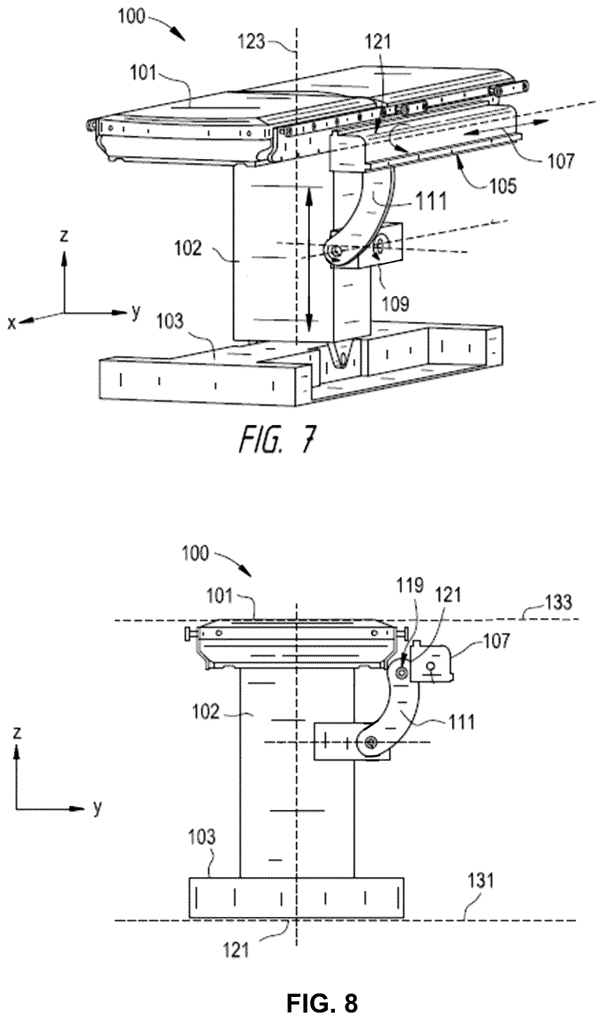

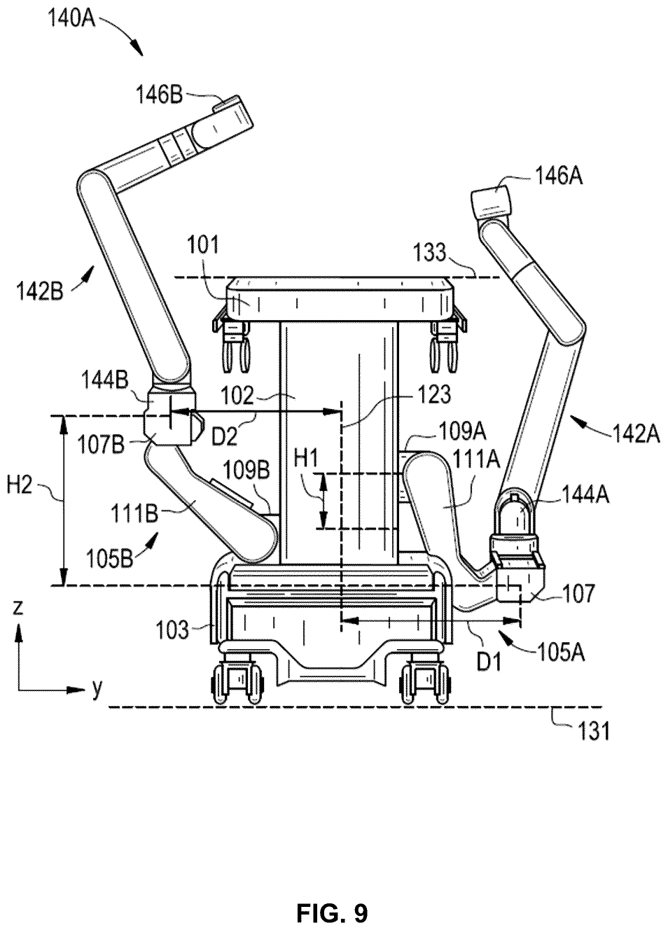

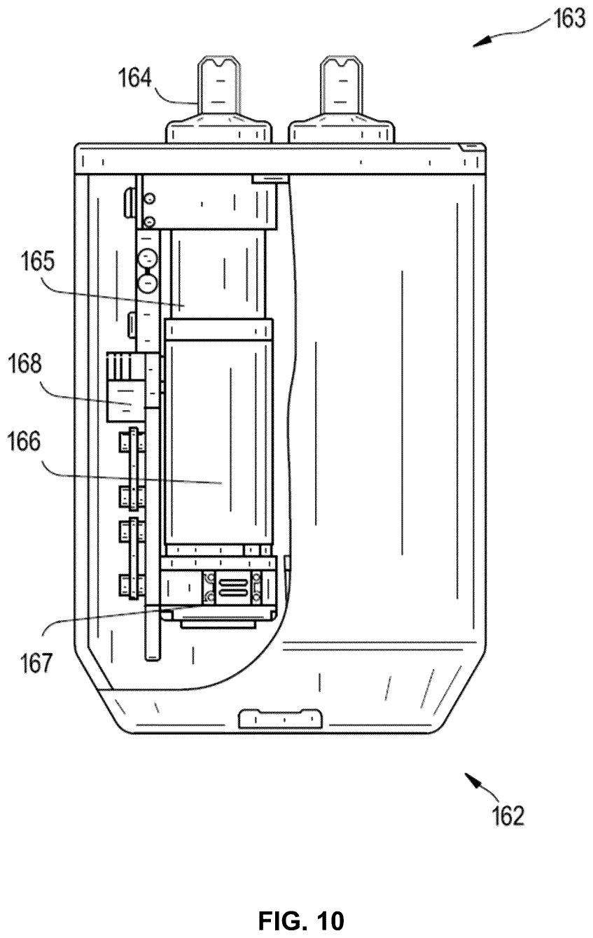

FIGS. 7 and 8 illustrate isometric and end views of an embodiment of a table-based surgical robotics system 100. The surgical robotics system 100 includes one or more adjustable arm supports 105 that can be configured to support one or more robotic arms (see, for example, FIG. 9) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 7 and 8, the arm support 105 is configured with four degrees of freedom, which are illustrated with arrows in FIG. 7. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 7 and 8 can comprise a table supported by a column 102 that is mounted to a base 103. The base 103 and the column 102 support the table 101 relative to a support surface. A floor axis 131 and a support axis 133 are shown in FIG. 8.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

FIG. 9 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporates electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

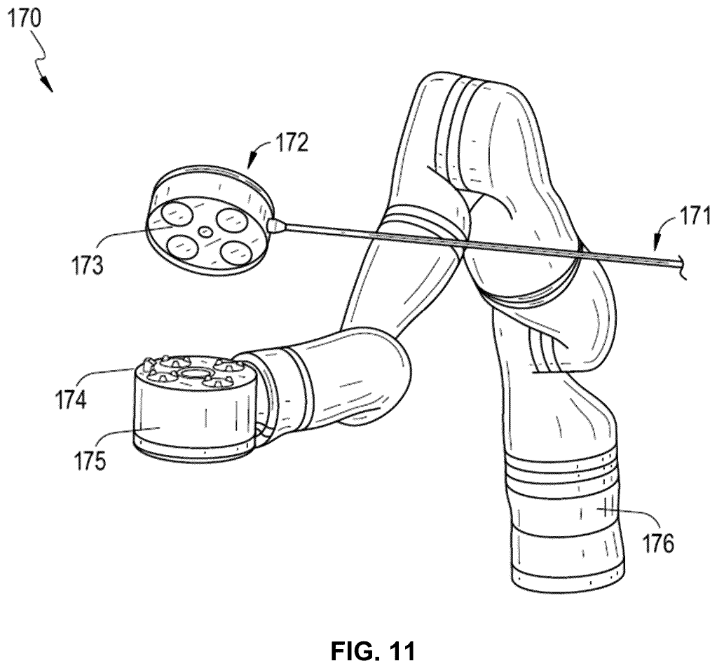

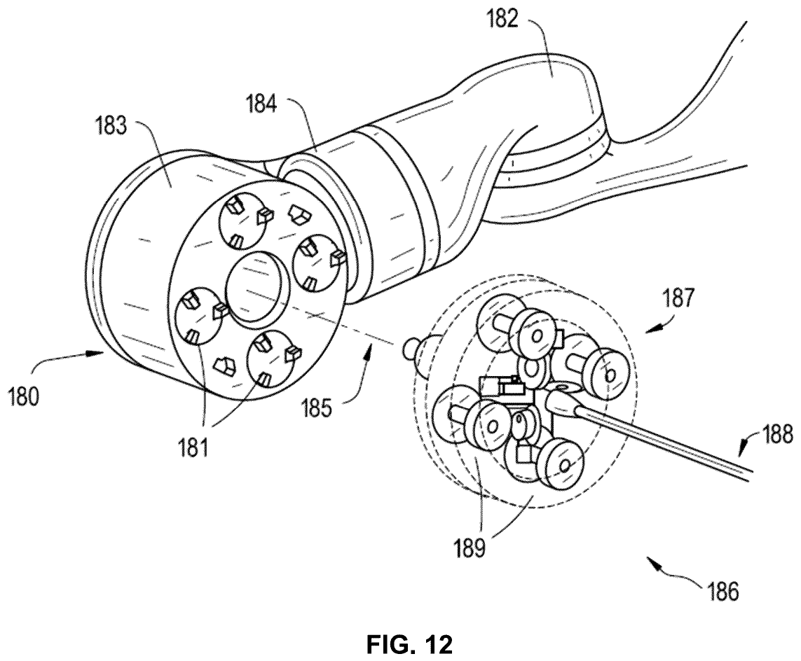

FIG. 10 illustrates an example instrument driver. Positioned at the distal end of a robotic arm, instrument driver 162 comprises of one or more drive units 163 arranged with parallel axes to provide controlled torque to a medical instrument via drive shafts 164. Each drive unit 163 comprises an individual drive shaft 164 for interacting with the instrument, a gear head 165 for converting the motor shaft rotation to a desired torque, a motor 166 for generating the drive torque, an encoder 167 to measure the speed of the motor shaft and provide feedback to the control circuitry, and control circuitry 168 for receiving control signals and actuating the drive unit. Each drive unit 163 being independent controlled and motorized, the instrument driver 162 may provide multiple (four as shown in FIG. 11) independent drive outputs to the medical instrument. In operation, the control circuitry 168 would receive a control signal, transmit a motor signal to the motor 166, compare the resulting motor speed as measured by the encoder 167 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

FIG. 11 illustrates an example medical instrument with a paired instrument driver. Like other instruments designed for use with a robotic system, medical instrument 170 comprises an elongated shaft 171 (or elongate body) and an instrument base 172. The instrument base 172, also referred to as an “instrument handle” due to its intended design for manual interaction by the physician, may generally comprise rotatable drive inputs 173, e.g., receptacles, pulleys or spools, that are designed to be mated with drive outputs 174 that extend through a drive interface on instrument driver 175 at the distal end of robotic arm 176. When physically connected, latched, and/or coupled, the mated drive inputs 173 of instrument base 172 may share axes of rotation with the drive outputs 174 in the instrument driver 175 to allow the transfer of torque from drive outputs 174 to drive inputs 173. In some embodiments, the drive outputs 174 may comprise splines that are designed to mate with receptacles on the drive inputs 173.

The elongated shaft 171 is designed to be delivered through either an anatomical opening or lumen, e.g., as in endoscopy, or a minimally invasive incision, e.g., as in laparoscopy. The elongated shaft 171 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 174 of the instrument driver 175. When designed for endoscopy, the distal end of a flexible elongated shaft may include a steerable or controllable bending section that may be articulated and bent based on torque received from the drive outputs 174 of the instrument driver 175.

Torque from the instrument driver 175 is transmitted down the elongated shaft 171 using tendons along the shaft 171. These individual tendons, such as pull wires, may be individually anchored to individual drive inputs 173 within the instrument handle 172. From the handle 172, the tendons are directed down one or more pull lumens along the elongated shaft 171 and anchored at the distal portion of the elongated shaft 171, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 173 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 171, where tension from the tendon cause the grasper to close.

In endoscopy, the tendons may be coupled to a bending or articulating section positioned along the elongated shaft 171 (e.g., at the distal end) via adhesive, control ring, or other mechanical fixation. When fixedly attached to the distal end of a bending section, torque exerted on drive inputs 173 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 171 to allow for controlled articulation in the desired bending or articulable sections.

In endoscopy, the elongated shaft 171 houses a number of components to assist with the robotic procedure. The shaft may comprise of a working channel for deploying surgical tools (or medical instruments), irrigation, and/or aspiration to the operative region at the distal end of the shaft 171. The shaft 171 may also accommodate wires and/or optical fibers to transfer signals to/from an optical assembly at the distal tip, which may include of an optical camera. The shaft 171 may also accommodate optical fibers to carry light from proximally-located light sources, such as light emitting diodes, to the distal end of the shaft.

At the distal end of the instrument 170, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera. In the example of FIG. 11, the drive shaft axes, and thus the drive input axes, are orthogonal to the axis of the elongated shaft.

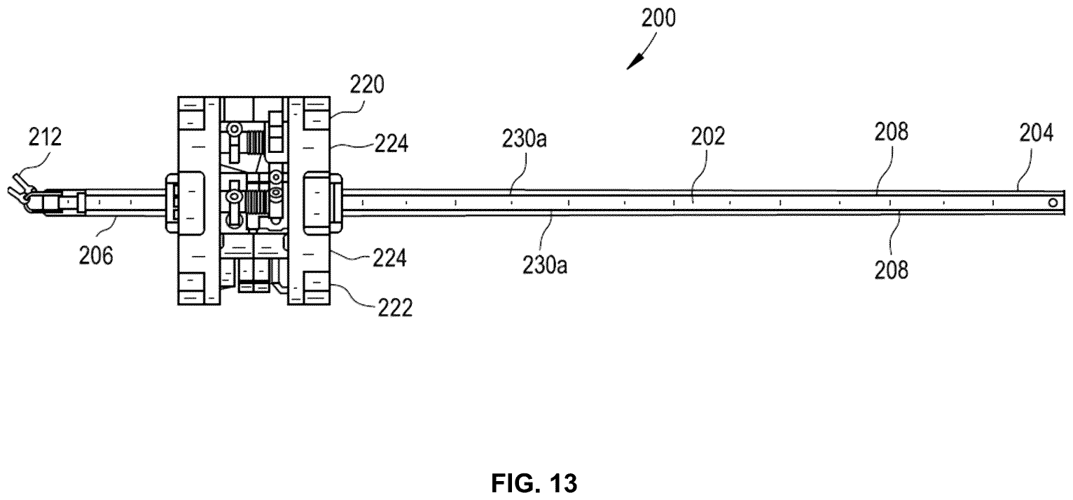

FIG. 12 illustrates an alternative design for an instrument driver and instrument where the axes of the drive units are parallel to the axis of the elongated shaft of the instrument. As shown, a circular instrument driver 180 comprises four drive units with their drive outputs 181 aligned in parallel at the end of a robotic arm 182. The drive units, and their respective drive outputs 181, are housed in a rotational assembly 183 of the instrument driver 180 that is driven by one of the drive units within the assembly 183. In response to torque provided by the rotational drive unit, the rotational assembly 183 rotates along a circular bearing that connects the rotational assembly 183 to the non-rotational portion 184 of the instrument driver. In other embodiments, the rotational assembly 183 may be responsive to a separate drive unit that is integrated into the non-rotatable portion 184, and thus not in parallel to the other drive units. The rotational mechanism 183 allows the instrument driver 180 to rotate the drive units, and their respective drive outputs 181, as a single unit around an instrument driver axis 185.

Like earlier disclosed embodiments, an instrument 186 may comprise an elongated shaft portion 188 and an instrument base 187 (shown with a transparent external skin for discussion purposes) comprising a plurality of drive inputs 189 (such as receptacles, pulleys, and spools) that are configured to receive the drive outputs 181 in the instrument driver 180. Unlike prior disclosed embodiments, instrument shaft 188 extends from the center of instrument base 187 with an axis substantially parallel to the axes of the drive inputs 189, rather than orthogonal as in the design of FIG. 11.

When coupled to the rotational assembly 183 of the instrument driver 180, the medical instrument 186, comprising instrument base 187 and instrument shaft 188, rotates in combination with the rotational assembly 183 about the instrument driver axis 185. Since the instrument shaft 188 is positioned at the center of instrument base 187, the instrument shaft 188 is coaxial with instrument driver axis 185 when attached. Thus, rotation of the rotational assembly 183 causes the instrument shaft 188 to rotate about its own longitudinal axis.

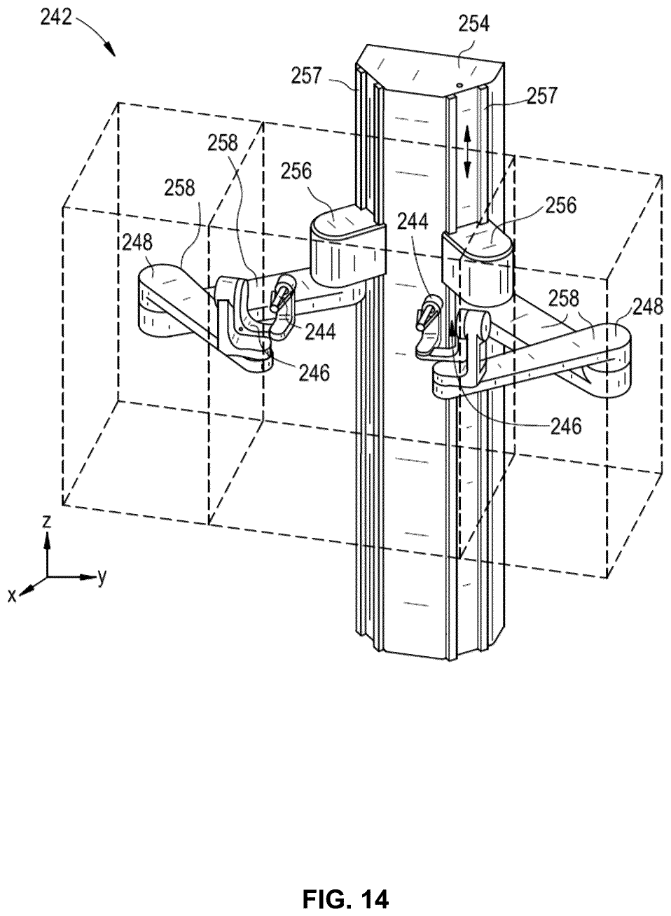

FIG. 13 illustrates an instrument having an instrument-based insertion architecture in accordance with some embodiments. The instrument 200 can be coupled to any of the instrument drivers discussed above. The instrument 200 comprises an elongated shaft 202, an end effector 212 connected to the shaft 202, and a handle 220 coupled to the shaft 202. The elongated shaft 202 comprises a tubular member having a proximal portion 204 and a distal portion 206. Manipulation of one or more cables 230a (e.g., via an instrument driver) results in actuation of the end effector 212.

The instrument handle 220, which may also be referred to as an instrument base, may generally comprise an attachment interface 222 having one or more mechanical inputs 224, e.g., receptacles, pulleys, or spools, that are designed to be reciprocally mated with one or more torque couplers on an attachment surface of an instrument driver.

In some embodiments, the instrument 200 comprises a series of pulleys or cables that enable the elongated shaft 202 to translate relative to the handle 220. In other words, the instrument 200 itself comprises an instrument-based insertion architecture that accommodates insertion of the instrument, thereby minimizing the reliance on a robot arm to provide insertion of the instrument 200. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

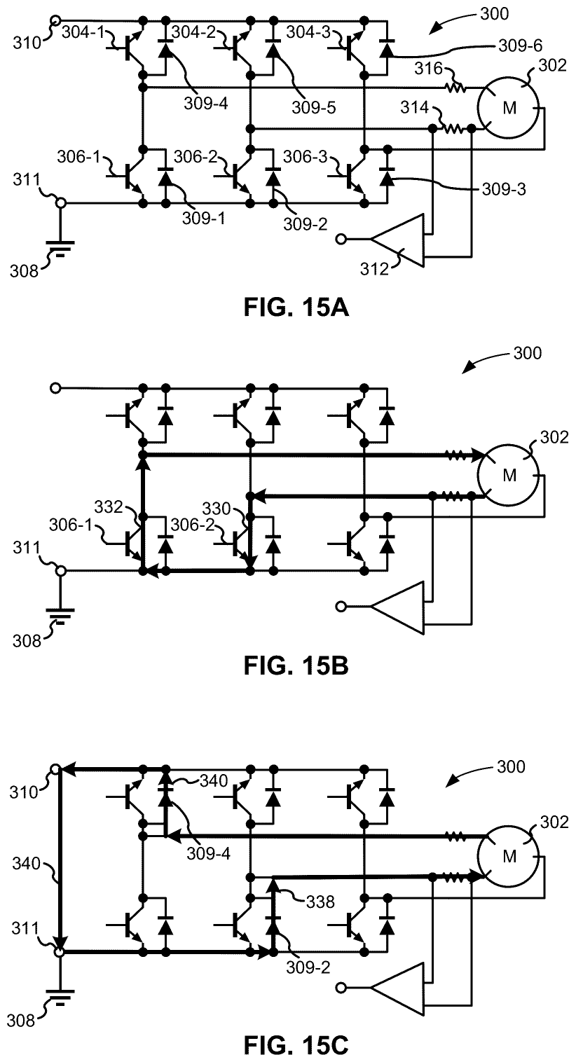

FIG. 14 is a perspective view of an embodiment of a controller 242. In the illustrated embodiment, the controller 242 is configured to allow manipulation of two medical instruments and includes two handles 244. Each of the handles 244 is connected to a gimbal 246. Each gimbal 246 is connected to a positioning platform 248.

As shown in FIG. 14, each positioning platform 248 includes a SCARA arm (selective compliance assembly robot arm) 258 coupled to a column 254 by a prismatic joint 256. The prismatic joints 256 are configured to translate along the column 254 (e.g., along rails 257) to allow each of the handles 244 to be translated in the z-direction, providing a first degree of freedom. The SCARA arm 258 is configured to allow motion of the handle 244 in an x-y plane, providing two additional degrees of freedom.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Shunting Circuits and Methods for Robotic Devices

Embodiments of the disclosure relate to circuits and methods for stopping a motor component of a robotic system or device. Robotic systems and devices, such as the systems described above, can advantageously include a shunting circuit or component to dynamically stop a motor (e.g., in response to a fault or emergency condition).

In many non-fault scenarios, joints of the robotic system are stopped under closed-loop control where the kinetic energy of the joints is converted by the motors into electrical energy that flows back onto the voltage bus. While this may raise the voltage of the bus, the bus can include resistors that allows at least some of the energy to be dissipated as heat to prevent large voltage spikes. In this way, the closed-loop control is able to control the current passing through the motor and can remove energy from the joints very quickly. In many cases, this is an efficient way to convert kinetic energy of the joints to electrical energy. Such closed-loop control may be used when a surgeon is performing a teleoperation and changes direction of the robotic component.

A challenge arises when a fault occurs at one of the sensors used to monitor and control operation of the motor, as the fault can prevent use of a closed-loop control. For example, closed-loop control may require knowledge of an electrical angle of the motor and the fault may prevent the closed-loop control from receiving information indicating the electrical angle. The shunting circuits and components described herein can provide a hardware control to stop a motor during an emergency stop (e-stop) without having to rely on software, or a sensor, that may be in an error state. Stopping a motor as quickly as possible during an e-stop helps ensure that a robotic arm and an associated medical tool do not continue to travel significantly so that patient safety can be enhanced.

Kinetic energy can be removed from the joints via transforming the kinetic energy into other forms of energy (e.g., energy generated through friction applied by brakes or thermal energy generated by a resistor). However, using brakes to stop joints may be inadequate, as the brakes alone may not provide sufficient friction to stop the joints quickly. Accordingly, it can be beneficial to transform at least some of the kinetic energy in the motor to thermal dissipation in a resistor. For example, the kinetic energy from the joints may be converted into electrical energy by the motor, and the electrical energy is then dissipated via one or more resistors.

One skilled in the art will appreciate that the systems and devices described herein can be applied in non-medical contexts as well. For example, the shunting circuits described herein may be implemented in robotic systems and devices used in non-medical environments.

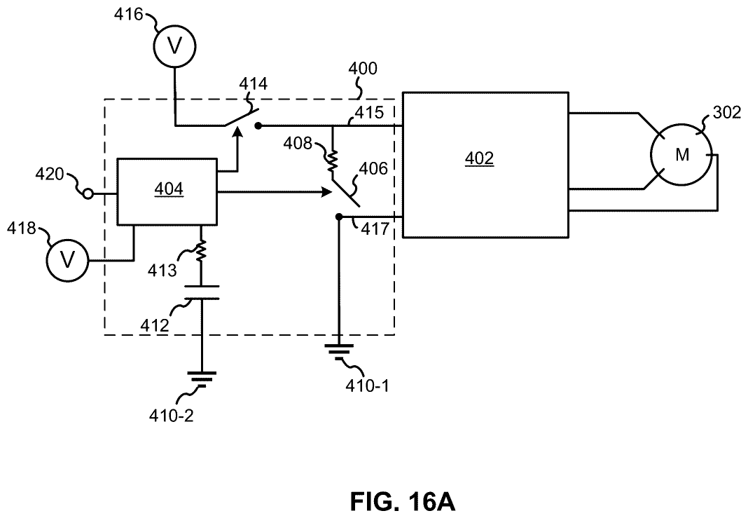

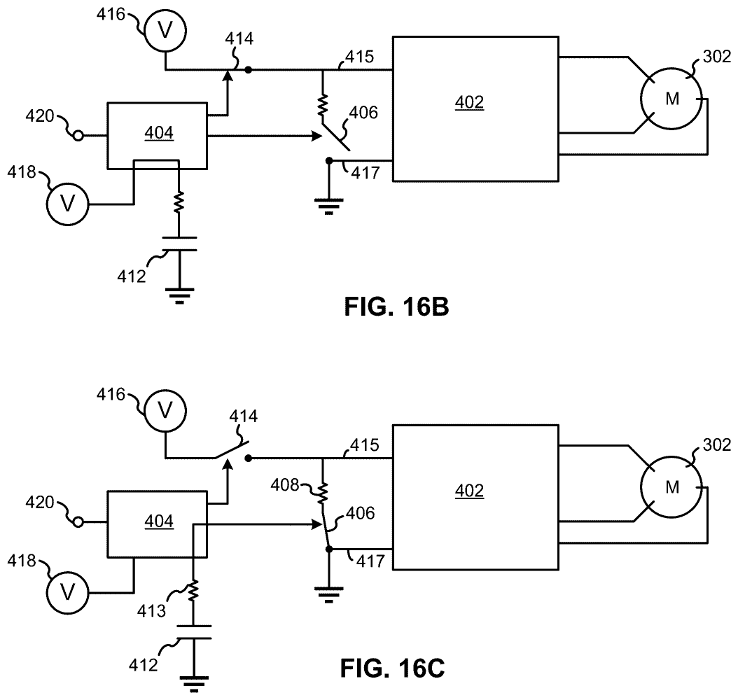

FIG. 15A illustrates a motor driver circuit 300 for a motor 302, according to some embodiments. In some embodiments, the motor 302 is coupled to the motor driver circuit 300 as shown in FIG. 15A and is not part of the motor driver circuit 300. In some embodiments, the motor 302 is an actuator. The motor driver circuit 300 includes three sets of transistor pairs (e.g., transistor pair 304-1 and 306-1, transistor pair 304-2 and 306-2, and transistor pair 304-3 and 306-3) for a three-phase motor 302. In some embodiments, the transistors 304 and 306 are field-effect transistors (FETs). Each transistor 304 is coupled between a voltage line 310 and a respective input of the motor 302. In some embodiments, each transistor 304 is a PMOS transistor. Each transistor 306 is coupled between a voltage line 311 and a respective input of the motor 302. In some embodiments, each transistor 306 is an NMOS transistor. In some embodiments, at least a subset of the transistors 304 and 306 are bipolar junction transistors. In some embodiments, the voltage line 311 is coupled to an electrical ground 308. In accordance with some embodiments, the voltage line 310 is a high voltage line (e.g., coupled to 48-volt voltage source) and the voltage line 311 is a low voltage line (e.g., coupled to an electrical ground). In some embodiments, the motor driver circuit 300 further includes a plurality of diodes 309 (e.g., diodes 309-1 through 309-6) with each diode coupled in parallel with a respective transistor (e.g., the diode 309-1 in parallel with the transistor 306-1, and the diode 309-4 in parallel with the transistor 304-1). In some embodiments, each diode 309 has the same properties (e.g., same forward voltages and same reverse voltages), whereas in other embodiments, at least a subset of the diodes 309 have differing properties (e.g., differing forward voltages and/or differing reverse voltages). In accordance with some embodiments, the motor driver circuit 300 further includes resistors 316 and 314 coupled to the motor 302. In accordance with some embodiments, the motor driver circuit 300 also includes a current sensor 312 (e.g., for monitoring the motor operation and/or phases) coupled to the resistor 314.

FIGS. 15B and 15C illustrate example shunting paths for the motor driver circuit 300 of FIG. 15A, according to some embodiments. FIG. 15B illustrates an example of gate shunting across the transistors 306-1 and 306-2. The transistors 306-1 and 306-2 in FIG. 15B are enabled (e.g., due to a voltage applied to the gate of each transistor) at the same time thereby creating a closed loop circuit between the motor 302 and the electrical ground 308. An example current flow is indicated by the arrows 330 and 332. In some situations, gate shunting as shown in FIG. 15B results in current spikes due to a small resistance in the circuit (e.g., due to the lack of additional resistors). Such a current spike may have an adverse effect on the motor or electronics of the robotic system.

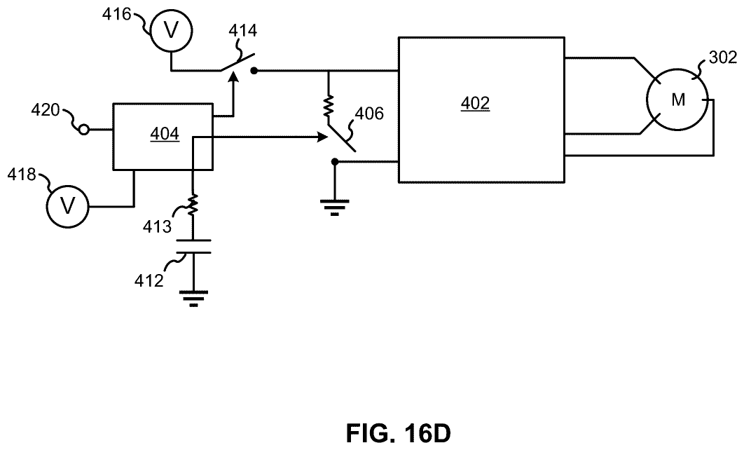

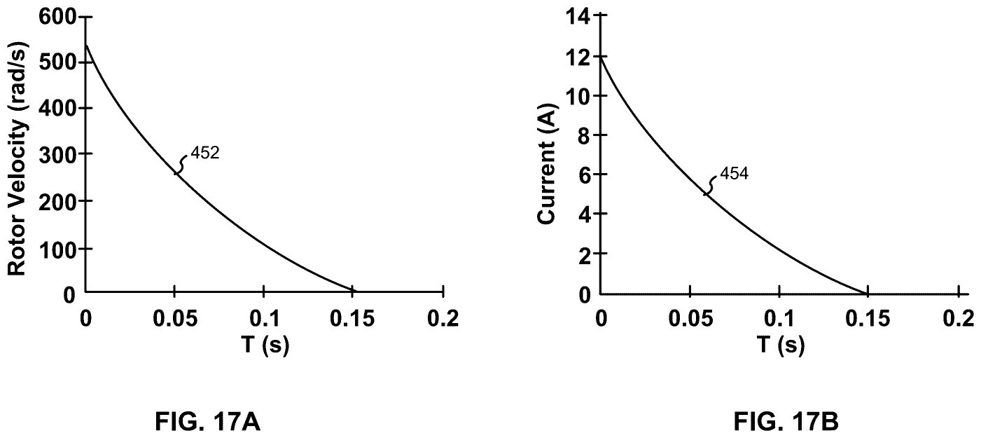

FIG. 15C illustrates an example of bus shunting across the diodes 309-2 and 309-4 by coupling the voltage line 310 to the voltage line 311 to create a closed loop circuit between the motor 302 and the electrical ground 308. An example current flow is indicated by the arrows 338 and 340. In the absence of additional resistance, the bus shunting shown in FIG. 15C may also result in current spikes. Configurations with an external resistor (e.g., the resistor 408 illustrated in FIG. 16A) added between the voltage 310 and the electrical ground 308 may limit or reduce the current flow (and reduce current spikes) during the shunting operation.

FIG. 16A illustrates a shunting circuit 400 for the motor 302, according to some embodiments. The shunting circuit 400 is coupled to the motor driver circuit 402 via lines 415 and 417. In some embodiments, the motor driver circuit 402 is an instance of the motor driver circuit 300 in FIG. 15A (e.g., the line 415 is coupled to the voltage line 310 and the line 417 is coupled to the voltage line 311). In FIG. 16A, the shunting circuit 400 includes switches 406 and 414, resistors 408 and 413, a capacitor 412, and a relay component 404. In some embodiments, the relay component 404 includes a plurality of relays (e.g., separate relays to control each of the switches 406 and 414). In some embodiments, the shunting circuit 400 includes a subset of switch 406, switch 414, resistor 408, resistor 413, a capacitor 412, or a relay component 404. In accordance with some embodiments, the shunting circuit 400 is coupled to a voltage source 416 (e.g., supplying a predefined voltage, such as 48 volts) and to the electrical grounds 410-1 and 410-2. In some embodiments, the electrical grounds 410-1 and 410-2 are the same electrical ground. In accordance with some embodiments, the relay component 404 is coupled to one or more control signals via the connection 420. In accordance with some embodiments, the relay component 404 is coupled to a voltage source 418 (e.g., a 24-volt voltage source). In some embodiments, the relay component 404 is coupled to a plurality of control signals and the connection 420 represents a bus connection (e.g., a parallel bus connection for independently transmitting a first control signal for controlling a first relay associated with the switch 406 and a second control signal for controlling a second relay associated with the switch 414). In some embodiments, the one or more control signals include one or more of: a test signal, a motor fault signal, a voltage source signal, or an emergency stop signal. In some embodiments, the relay component 404 is, or includes, a solid-state relay (SSR), such as an active-low solid-state relay.

In some embodiments, the resistor 408 is adapted (e.g., sized) to have a resistance sufficient to reduce the current flowing through the motor during the shunting operation (e.g., the current shown in FIG. 15C). In some configurations, the resistor 408 prevents the current from damaging the motor or other electronics. In some embodiments, the resistor 408 is adapted (e.g., sized) to have a minimum resistance sufficient to prevent the current flowing through the motor during the shunting operation from damaging the motor or other electronics. In some configurations, by having a minimum sufficient resistance, the resistor 408 is able to protect the motor and electronics without introducing undue delay in stopping the motor during the shunting operation. In some embodiments, the resistor 408 has a resistance in the range of 1 ohm to 50 ohms (e.g., 5, 10, 15, 20, 25, 30, 35, 40, or 45 ohms). In some embodiments, the switch 414 is, or includes, a transistor (e.g., a FET) and the relay component 404 includes a transistor driver component that controls opening and closing of the switch 414 (e.g., via a voltage applied to the gate of the transistor).

FIGS. 16B-16D illustrate example operating states for the shunting circuit 400 of FIG. 16A, according to some embodiments. FIG. 16B illustrates a non-shunting operating state for the shunting circuit 400. In accordance with some embodiments, the non-shunting operating state represents a normal operating state for the motor 302. As illustrated in FIG. 16B, in the non-shunting operating state, the switch 414 is closed (e.g., allowing current to flow between the voltage source 416 and the motor driver circuit 402) and the switch 406 is open (e.g., preventing current from flowing between the lines 415 and 417). In accordance with some embodiments, in the non-shunting operating state, the capacitor 412 is coupled via the relay component 404 to the voltage source 418 (e.g., so as to charge the capacitor 412). In some embodiments, the voltage source 418 is, or includes, a capacitor.

FIG. 16C illustrates a shunting operating state for the shunting circuit 400. As illustrated in FIG. 16C, in the shunting operating state, the switch 414 is open (e.g., preventing current from flowing between the voltage source 416 and the motor driver circuit 402) and the switch 406 is closed (e.g., allowing current to flow between the lines 415 and 417). In some embodiments, the shunting circuit 400 transitions to the shunting operating state in response to one or more of the control signal(s) being active. In some embodiments, the control signal(s) are active-low control signals (e.g., a control signal transitioning to a low voltage (e.g., 0 volts) represents an active signal). As illustrated in FIG. 16C, in the shunting operating state, the motor driver circuit 402 is bus shunted across the resistor 408, thereby converting kinetic energy from the motor 302 to thermal energy (e.g., heat dissipation) using the resistor 408.

As also illustrated in FIG. 16C, in the shunting operating state, the capacitor 412 is coupled to the switch 406 via the relay component 404 and the resistor 413. In some embodiments, the capacitor 412 is adapted (e.g., sized) to have a capacitance that, in combination with the resistance of the resistor 413, dictates an RC time constant for how long a shunting operation occurs (e.g., how long the switch 406 remains closed) after a fault is detect. In some embodiments, the capacitor 412 has a capacitance in the range of 1 microfarad to 100 microfarads (e.g., 10, 20, 30, 40, 50, 60, 70, 80, or 90 microfarads). In some embodiments, the resistor 413 has a resistance in the range of 10 kiloohms to 100 kiloohms (e.g., 10, 20, 30, 40, 50, 60, 70, 80, or 90 kiloohms). In some embodiments, the resistor 413 and the capacitor 412 are adapted to have an RC time constant in the range of 0.1 seconds to 2 seconds (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, or 1.9 seconds).

FIG. 16D illustrates a backdrivable operating state for the shunting circuit 400. As illustrated in FIG. 16D, in the backdrivable operating state, the switch 414 is open (e.g., preventing current from flowing between the voltage source 416 and the motor driver circuit 402) and the switch 406 is open (e.g., preventing current from flowing between the lines 415 and 417). In some embodiments, the shunting circuit 400 transitions to the backdrivable operating state after being in the shunting operating state. In accordance with some embodiments, the backdrivable operating state corresponds to a low stiffness state for a joint driven by the motor 302, thereby easing manual manipulation of the joint by an operator of the robotic system. For example, opening the switch 406 reduces a resistance against manual manipulation of the joint (e.g., by an operator of the robotic system). In some embodiments, the shunting circuit 400 transitions to the backdrivable operating state after being in the shunting operating state for a set amount of time. In some embodiments, the set amount of time is based on the resistor 413 and the capacitor 412. The resistor 413 has a resistance value (R) and the capacitor 412 has a capacitance value (C). In some embodiments, the set amount of time corresponds to an RC time constant of the resistor 413 and the capacitor 412. In some embodiments, the shunting circuit 400 includes one or more additional resistances coupled to the capacitor 412 (not shown) and the RC time constant is based on a combination of resistances from the resistor 413 and the one or more additional resistors.

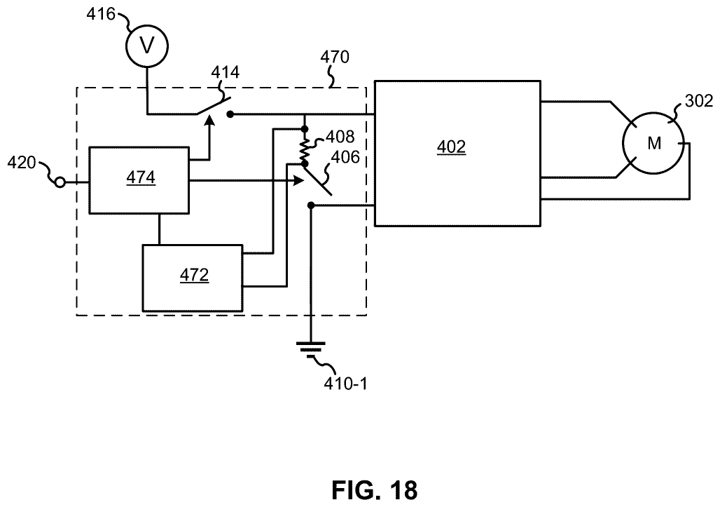

FIGS. 17A and 17B illustrate example shunting characteristics for the shunting circuit 400 of FIG. 16A, according to some embodiments. FIG. 17A shows an example of a change in the rotor velocity of the motor 302 over time (represented by the line 452) during the shunting operating state of FIG. 16C. FIG. 17B shows an example of a change in current flowing through the motor 302 over time (represented by the line 454) during the shunting operating state of FIG. 16C. In the example illustrated in FIGS. 17A-17B, the shunting circuit 400 switches from the non-shunting operating state to the shunting operating state at zero seconds. In accordance with some embodiments, the shunting circuit 400 switches from the shunting operating state to the backdrivable operating state as the rotor velocity approaches zero radians per second (e.g., at 0.15 seconds). In some embodiments, the shunting circuit 400 switches from the shunting operating state to the backdrivable operating state in accordance with a determination that the rotor velocity is below a predefined velocity threshold or the current is below a predefined current threshold. In some embodiments, the predefined velocity threshold is non-zero and the predefined current threshold is non-zero. In accordance with some embodiments, the resistor 413 and the capacitor 412 of the shunting circuit 400 define an RC time constant that dictates when the shunting circuit 400 transitions from the shunting operating state to the backdrivable state. For example, the capacitor 412 is charged via the relay component 404 (e.g., during the non-shunting operating state) and is used to close the switch 406 during the shunting operating state. In this example, once the capacitor 412 is sufficiently drained, the switch 406 reopens (e.g., the gate of the switch 406 is no longer driven to keep the switch 406 in the closed state).

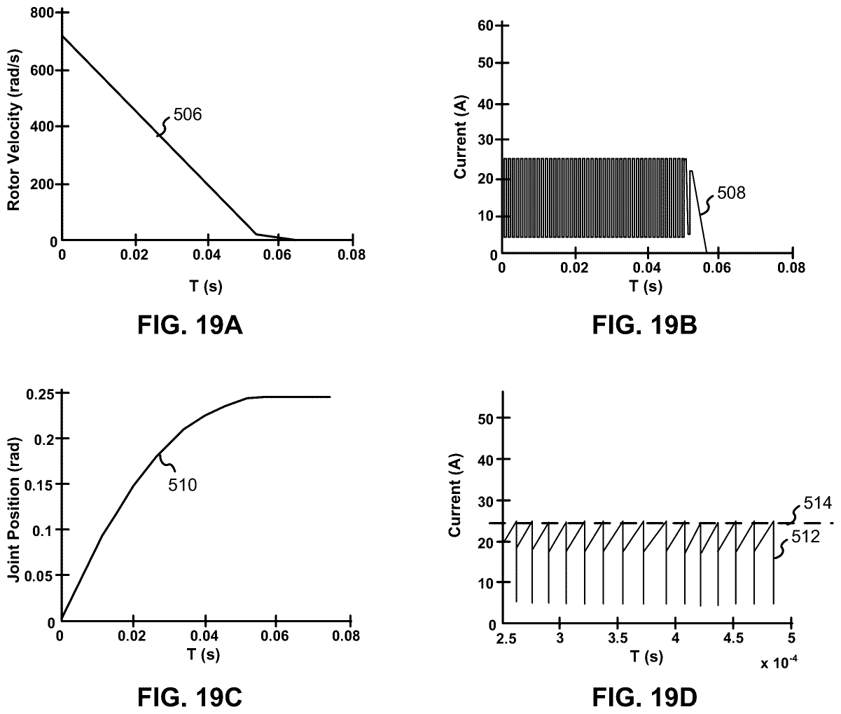

FIG. 18 illustrates a shunting circuit 470 for the motor 302, according to some embodiments. The shunting circuit 470 is similar to the shunting circuit 400 (shown in FIG. 16A) except that the shunting circuit 470 includes a current sensor 472 and does not include the resistor 413 or the capacitor 412. Additionally, the relay component 404 of the shunting circuit 400 is replaced with a relay component 474 in the shunting circuit 470. In some embodiments, the relay component 474 is an instance of the relay component 404. In accordance with some embodiments, the relay component 474 is configured to chopper shunt the motor driver circuit 402 by opening and closing the switch 406 during the shunting operating state (e.g., the relay component 474 repeats opening and closing the switch 406 for chopper shunting operation during the shunting operating state). For example, at a first time (e.g., upon transitioning to the shunting operating state), the relay component 474 causes the switch 406 to close. At a second time, in accordance with a current across the resistor 408 reaching a current threshold (e.g., as measured by the current sensor 472), the relay component 474 causes the switch 406 to open. At a third time (e.g., a set amount of time after the second time) the relay component 474 causes the switch 406 to close again. In some embodiments, the relay component 474 repeats (and alternates) the opening and closing of the switch 406 until the rotor velocity of the motor 302 is reduced to a velocity threshold or below. In some embodiments, the shunting circuit 470 does not include the current sensor 472 (e.g., the relay component 474 receives a current indicator from another component, such as the motor driver circuit 402). In some embodiments, the current sensor 472 determines the current provided to the resistor 408 by measuring a voltage across the resistor 408 (and based on a predefined or known resistance of the resistor 408).

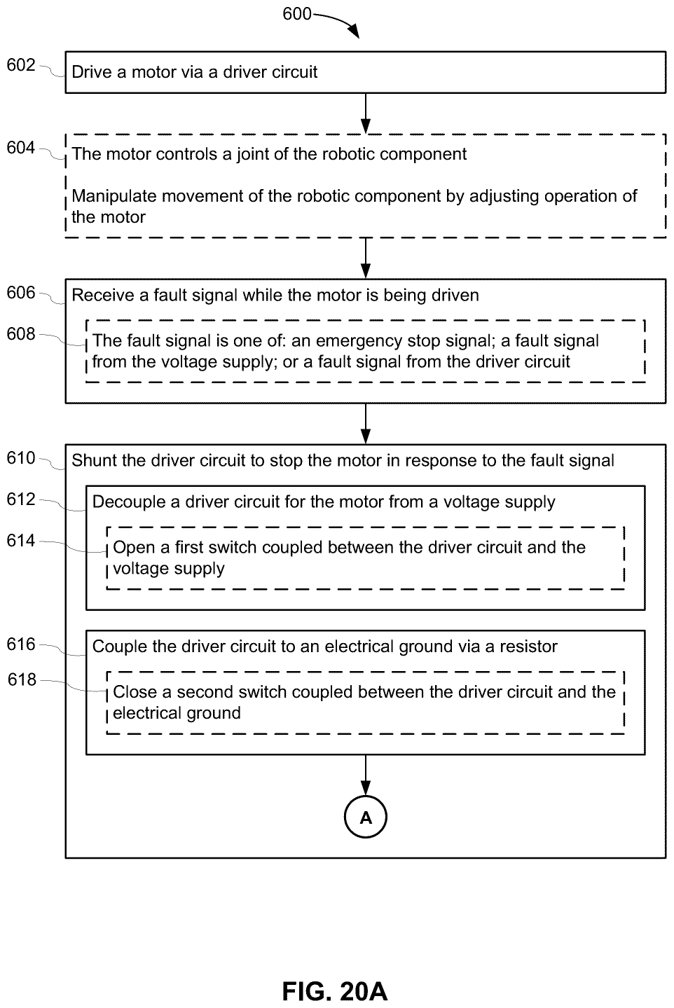

FIGS. 19A-19D illustrate example shunting characteristics for the shunting circuit 470 of FIG. 18, according to some embodiments. FIG. 19A shows an example of a change in the rotor velocity of the motor 302 over time (represented by the line 506) during the shunting operating state as the relay component 474 chopper shunts the motor driver circuit 402. FIG. 19B shows an example of a change in current through the motor 302 over time (represented by the line 508) during the shunting operating state as the relay component 474 chopper shunts the motor driver circuit 402. FIG. 19C shows an example of a change in position of the joint driven by the motor 302 over time (represented by the line 510) during the shunting operating state as the relay component 474 chopper shunts the motor driver circuit 402. FIG. 19D shows another example of a change in current through the motor 302 over time (represented by the line 512) during the shunting operating state (at a reduced timescale as compared to FIG. 19B). In accordance with some embodiments, the dashed line 514 in FIG. 19D represents the current threshold described above with respect to FIG. 18. For example, the local maximums (peaks) in the line 512 represent the current meeting the current threshold where the relay component 474 causes the switch 406 to open. In this example, the current increasing from the local minimums (valleys) in the line 512 represent the relay component 474 causing the switch 406 to close again.

In the example illustrated in FIGS. 19A-19D, the shunting circuit 470 switches from the non-shunting operating state to the shunting operating state at zero seconds. In accordance with some embodiments, the shunting circuit 470 switches from the shunting operating state to the backdrivable operating state as the rotor velocity approaches zero radians per second (e.g., at 0.06 seconds). In some circumstances, chopping shunting allows for shunting of the motor using a smaller resistor as compared to non-chopper shunting (e.g., the resistor 408 may have less resistance in the shunting circuit 470 of FIG. 18 than in the shunting circuit 400 of FIG. 16A.

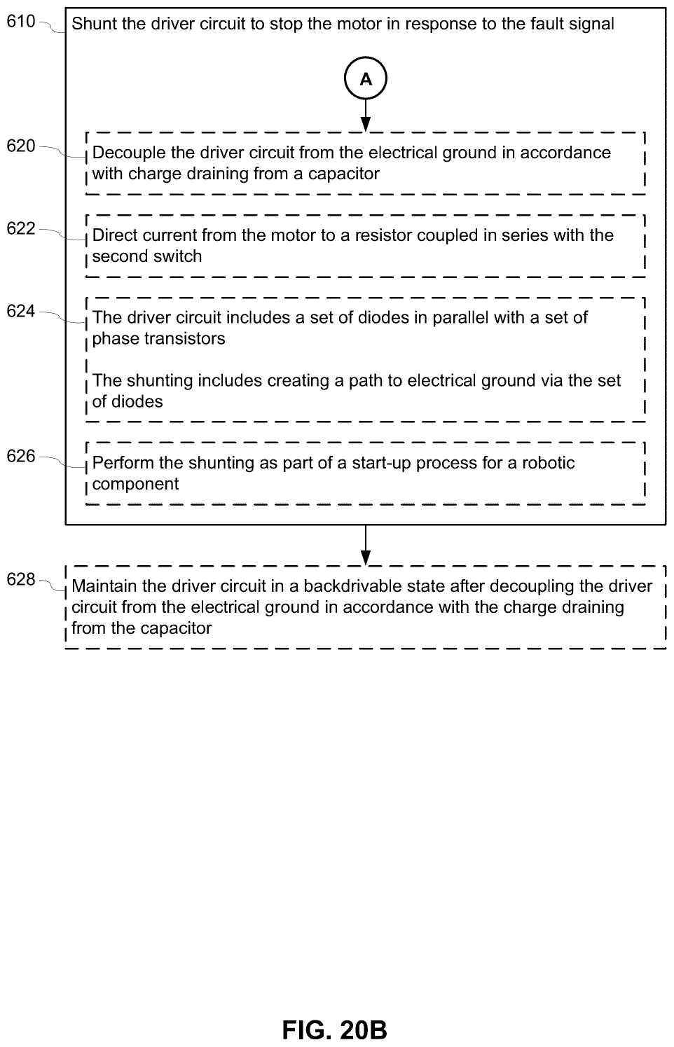

FIGS. 20A and 20B are a flowchart illustrating a method 600 for shunting a motor in accordance with some embodiments. The method 600 is performed at a medical system (e.g., the medical system 36) having one or more processors (e.g., the processor(s) 280) and memory (e.g., the memory 282). In some embodiments, the memory (e.g., the memory 282) stores instructions for execution by the one or more processor (e.g., the processor(s) 280). In some embodiments, the medical system includes a robotic arm (e.g., is a robotically-enabled medical system). In some embodiments, the robotic arm is coupled to a medical instrument.

In some embodiments, the medical system includes: (i) a motor (e.g., the motor 302) of a robotic component (e.g., a robotic arm); (ii) a driver circuit (e.g., the driver circuit 402) coupled to the motor; and (iii) a shunting circuit (e.g., the shunting circuit 400) coupled to the driver circuit and configured to shunt the driver circuit for a set amount of time to stop the motor. In some embodiments, the shunting circuit includes a capacitor (e.g., the capacitor 412) and a first resistor (e.g., the resistor 413), the capacitor and first resistor having (e.g., characterized by, or defining) an RC time constant, where the set amount of time corresponds to the RC time constant.

In some embodiments, a medical system includes: (i) a motor (e.g., the motor 302) of a robotic component; (ii) a driver circuit (e.g., the driver circuit 402) coupled to the motor; (iii) a shunting circuit (e.g., the shunting circuit 470) coupled to the driver circuit and configured to shunt the driver circuit to stop the motor. In some embodiments, the shunting circuit includes: (a) a first switch (e.g., the switch 414) coupling the driver circuit to a voltage source; (b) a second switch (e.g., the switch 406) coupling the driver circuit to an electrical ground; (c) a current sensor (e.g., the current sensor 472) coupled to the driver circuit and configured to measure a current in the driver circuit; and (d) a control component (e.g., the relay component 474) coupled to the current sensor, the first switch, and the second switch. In some embodiments, the control component is configured to, in response to a fault signal: (i) open the first switch; and (ii) selectively open and close the second switch to maintain the current in the driver circuit within a preset range while stopping the motor.

In some embodiments, a medical system includes: (i) a motor (e.g., the motor 302) of a robotic component; (ii) a driver circuit (e.g., the driver circuit 402) coupled to the motor; (iii) a shunting circuit (e.g., the shunting circuit 400) coupled to the driver circuit. In some embodiments, the shunting circuit includes: (a) a switch (e.g., the switch 406) electrically coupled between a high voltage line of the driver circuit (e.g., the voltage line 310) and an electrical ground (e.g., the electrical ground 410-1); (b) a resistor (e.g., the resistor 408) electrically coupled (e.g., in series to the switch) between the high voltage line of the driver circuit and an electrical ground; and (c) a capacitor (e.g., the capacitor 412) coupled to the switch (e.g., to a gate of a transistor of the switch) for shunting the driver circuit over a set amount of time (e.g., by keeping the switch closed for the set amount of time while a charge in the capacitor drains).

The medical system drives (602) a motor (e.g., the motor 302) via a driver circuit (e.g., the driver circuit 300). In some embodiments, the medical system drives the motor using the processor(s) 280 and/or control circuitry coupled to the driver circuit (e.g., control circuitry coupled to the gates of the transistors 304 and 306). In some embodiments, the motor is a multi-phase motor (e.g., a 3-phase motor) and the driver circuit is configured to drive each of the individual phases of the multi-phase motor.

In some embodiments, the motor controls (604) a joint of the robotic component, where movement of the robotic component is manipulated by adjusting operation of the motor. In some embodiments, the robotic component is an instance of any one of the robotic arms described previously (e.g., robotic arm 12, 39, 50, 142, 176, 182, or 258). In some embodiments, the joint of the robotic component is an instance of any one of the joints described previously (e.g., the joints 24 and 256).

The medical system receives (606) a fault signal while the motor is being driven. In some embodiments, the fault signal is received at a shunting circuit (e.g., the shunting circuit 400). In some embodiments, the fault signal is received at a relay component of the shunting circuit (e.g., the relay component 404). In some embodiments, the fault signal is an active-low signal (e.g., a transition to a low voltage (e.g., 0 volts) represents an active signal). In some embodiments, the fault signal is received by the shunting circuit at an input terminal (e.g., the connection 420). In some embodiments, the fault signal is received from one or more processors 280 or one or more sensors of the medical system.

In some embodiments, the fault signal is (608) one of: an emergency stop signal, a fault signal from a voltage source, or a fault signal from the driver circuit. In some embodiments, the voltage source is a voltage source for the motor driver circuit (e.g., the voltage source 416). In some embodiments, the voltage source is a voltage source for control circuitry of the medical system (e.g., a 24-volt, 5-volt, or 3-volt voltage source). In some embodiments, the fault signal corresponds to a testing request for the shunting circuit (e.g., prior to use of the medical system in a medical operation).

The medical system shunts (610) the driver circuit to stop the motor in response to the fault signal. For example, the medical system couples a high voltage line of the driver circuit (e.g., the voltage line 310) to a low voltage line (e.g., the voltage line 311) to create a closed circuit to an electrical ground (e.g., the electrical ground 308) (e.g., via the resistor 408).

Shunting the driver circuit includes decoupling (612) a driver circuit for the motor from a voltage source. For example, the switch 414 of the shunting circuit 400 is opened to disconnect the line 415 from the voltage source 416. In some embodiments, the driver circuit includes a set of diodes (e.g., the diodes 309) in parallel with a set of phase transistors (e.g., the transistors 304 and 306), and the shunting circuit shunts the driver circuit by creating a path to electrical ground via the set of diodes (e.g., the shunting circuit bus shunts the motor).

In some embodiments, the medical system opens (614) a first switch (e.g., the switch 414) coupled between the driver circuit and the voltage source to decouple the driver circuit. In some embodiments, the first switch is, or includes, a transistor (e.g., a FET) and the medical system opens the first switch using a transistor driver circuit (e.g., coupled to the gate of the transistor). In some embodiments, the medical system opens the first switch using a relay component (e.g., the relay component 404). In some embodiments, the relay component is coupled to a gate of a transistor of the first switch.

Shunting the driver circuit includes coupling (616) the driver circuit (e.g., a high voltage line 415 of the driver circuit) to an electrical ground (e.g., the electrical ground 410-1) via a resistor (e.g., the resistor 408). In some embodiments, coupling the driver circuit to the electrical ground creates a closed circuit that involves the motor. In some embodiments, the resistor is adapted (e.g., sized) based on characteristics of the motor (e.g., a resistance of the motor and a maximum velocity of the motor).

In some embodiments, the medical system closes (618) a second switch (e.g., the switch 406) coupled between the driver circuit and the electrical ground to couple the driver circuit to the electrical ground. In some embodiments, the second switch is, or includes, a transistor (e.g., a FET). In some embodiments, the medical system closes the second switch using a relay component (e.g., the relay component 404). In some embodiments, the relay component is coupled to a gate of a transistor of the second switch.

In some embodiments, the medical system shunts the driver circuit using a shunting circuit (e.g., the shunting circuit 400). In some embodiments, the shunting circuit includes: (i) a first switch (e.g., the switch 414) coupled between the driver circuit and a voltage source (e.g., the voltage source 416); (ii) a second switch (e.g., the switch 406) coupled between the driver circuit and an electrical ground (e.g., the electrical ground 410-1); and (iii) a relay component (e.g., the relay component 404) coupled to a plurality of fault signal inputs (e.g., via the connection 420). In some embodiments, the shunting circuit includes a subset of the first switch, the second switch, and the relay component. In some embodiments, the relay component 404 is configured to: (a) in response to a fault signal at one of the plurality of fault signal inputs, open the first switch and close the second switch; and (b) after the set amount of time from receiving the fault signal, close the first switch and open the second switch. In some embodiments, the relay component is coupled to the plurality of fault signal inputs directly. In some embodiments, the relay component is coupled to the plurality of fault signal inputs indirectly, e.g., via a multiplexer or a signal combiner, such as a logical AND (or NAND) or a logical OR (or NOR) gate. In some embodiments, the first switch and the second switch each include a transistor (e.g., a FET). In some embodiments, the relay component includes a solid-state relay (e.g., an active-low solid-state relay). In some embodiments, the plurality of fault signal inputs includes at least one of: an emergency stop signal; a fault signal from the voltage source; or a fault signal from the driver circuit.

In some embodiments, the medical system decouples (620) the driver circuit from the electrical ground in accordance with charge draining from a capacitor (e.g., the capacitor 412). For example, the switch 406 includes an n-type (NMOS) transistor that operates in an “on state” (e.g., as a closed switch) while a charge at the gate of the NMOS transistor is sufficiently high. In this example, the capacitor is charged prior to the medical system receiving the fault and the capacitor maintains the NMOS transistor in the on state until sufficient charge drains from the capacitor.

In some embodiments, the capacitor (e.g., the capacitor 412) is coupled with one or more resistors (e.g., the resistor 413), and the capacitor and the one or more resistors are characterized by an RC time constant. In some embodiments, the RC time constant is between 0.5 seconds and 5 seconds. In some embodiments, the time constant is selected to stop the motor based on a worst-case scenario (e.g., max velocity movement when fault occurs). In some embodiments, the capacitor has a capacitance in the range of 1 microfarad to 100 microfarads. In some embodiments, the one or more resistors have a resistance in the range of 10 kiloohms to 100 kiloohms (e.g., 50 kiloohms).

In some embodiments, the medical system decouples the driver circuit from the electrical ground in accordance with charge draining from a capacitor. For example, the switch 406 includes an p-type (PMOS) transistor that is operates in an “on state” (e.g., as a closed switch) while a charge at the gate of the PMOS transistor is sufficiently low. In this example, the capacitor begins to charge when the medical system enters the shunting operating state, and the capacitor maintains the PMOS transistor in the on state until sufficient charge builds up in the capacitor.

In some embodiments, the medical system directs (622) current from the motor to a resistor (e.g., the resistor 408) coupled in series with the second switch (e.g., the switch 406). In some embodiments, the shunting circuit includes the resistor (e.g., the resistor 408) coupled in series with the second switch. In some embodiments, the resistor is sized to provide a resistance sufficient to prevent the motor from overheating without introducing undue delay in stopping the motor.

In some embodiments, the driver circuit includes (624) a set of diodes (e.g., the diodes 309) in parallel with a set of phase transistors (e.g., the transistors 304 and 306), and the shunting includes creating a path to electrical ground via the set of diodes (e.g., as illustrated in FIG. 15C).

In some embodiments, the medical system performs (626) the shunting as part of a start-up process for a robotic component. In some embodiments, the medical system is configured to test the shunting circuit as part of a start-up (power-on) process. In some embodiments, the medical system performs the shunting as part of a testing sequence prior to performing a medical operation (e.g., a surgical operation) on a patient. In some embodiments, the medical system performs the shunting on a periodic basis to test operation of the shunting circuit and/or motor driver circuit (e.g., daily, weekly, or monthly).

In some embodiments, the medical system maintains (628) the driver circuit in a backdrivable state after decoupling the driver circuit from the electrical ground in accordance with the charge draining from the capacitor. In some embodiments, the medical system maintains the driver circuit in the backdrivable state by decoupling a voltage line (e.g., a high voltage line) of the driver circuit from any voltage source or electrical ground (e.g., as illustrated in FIG. 16D). In some embodiments, the medical system maintains the driver circuit in the backdrivable state after the capacitor (e.g., the capacitor 412) drains sufficient charge to be unable to maintain the switch 406 in the closed state. In some embodiments, the medical system maintains the driver circuit in the backdrivable state until the fault signal is cleared, or overridden, or the medical system is reset or restarted (e.g., after the source of the fault signal is addressed).

In some embodiments, a method for stopping a motor (e.g., the motor 302) of a robotic component includes: (i) driving the motor via a driver circuit (e.g., the driver circuit 402); (ii) while the motor is being driven, receiving a fault signal (e.g., receiving the fault signal at the relay component 404); (iii) in response to the fault signal, shunting the driver circuit (e.g., using the shunting circuit 400) to stop the motor, including: (a) decoupling a driver circuit for the motor from a voltage source (e.g., decoupling a high voltage line of the driver circuit from the voltage source); and (b) coupling the driver circuit to an electrical ground via a resistor (e.g., coupling the high voltage line of the driver circuit to the electrical ground 410-1), where the driver circuit is decoupled from the electrical ground in accordance with charge draining from a capacitor (e.g., the capacitor 412).

In some embodiments, a method for stopping a motor (e.g., the motor 302) of a robotic component includes: (i) driving the motor via a driver circuit (e.g., the driver circuit 402); (ii) while the motor is being driven, receiving a fault signal (e.g., receiving the fault signal at the relay component 474); (iii) in response to the fault signal, shunting the driver circuit to stop the motor (e.g., via the shunting circuit 470), including: (a) decoupling the driver circuit for the motor from a voltage source (e.g., the voltage source 416); and (b) selectively coupling a high voltage line of the driver circuit to an electrical ground (e.g., the electrical ground 410-1) until the motor stops. In some embodiments, the selective coupling includes: (i) monitoring a current in the driver circuit (e.g., via the current sensor 472); (ii) in accordance with the current exceeding a first predetermined threshold (e.g., the dashed line 514), decoupling the driver circuit from the electrical ground; and (iii) in accordance with the current going below a second predetermined threshold, recoupling the driver circuit to the electrical ground (e.g., as illustrated in FIGS. 19A-19D).

### 3. Implementing Systems and Terminology

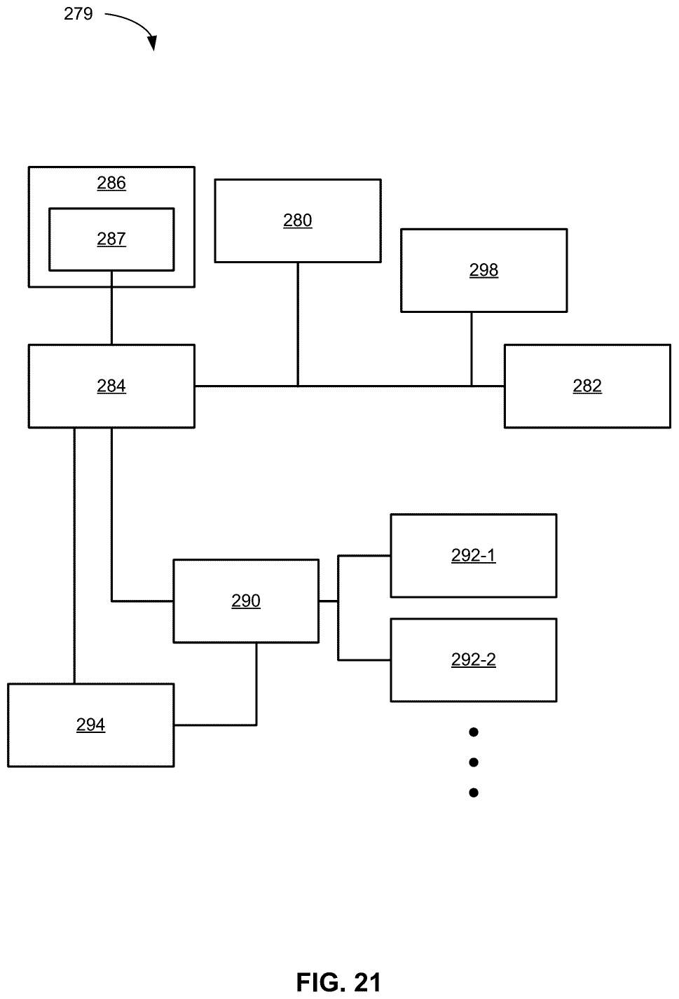

FIG. 21 is a schematic diagram illustrating electronic components of a system 279 (e.g., a medical and/or robotic system). In some embodiments, the system 279 is an instance of one of the systems 10, 36, 47, 100, and 140A. The system 279 includes one or more processors 280, which are in communication with a computer-readable storage medium of memory 282 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 20A-20B). In some embodiments, the one or more processors 280 are in communication with a user interface 298. In some embodiments, the user interface 298 includes one or more a human-machine interface (HMI) components, such as a console (e.g., the console 31), a display (e.g., the touchscreen 26), a speaker, and/or a warning light (e.g., an LED).

The one or more processors 280 are also in communication with an input/output controller 284 (via a system bus or any suitable electrical circuit). The input/output controller 284 receives user input from the input device 286 (e.g., the robotic arm 182, or the instrument 200) and, optionally, sensor data from one or more sensors 287, and relays the data and user input to the one or more processors 280. The input/output controller 284 also receives instructions and/or data from the one or more processors 280 and relays the instructions and/or data to one or more motors, such as motors 292-1 and 292-2 (e.g., actuators and motors for driving robotic arms of the system). In some embodiments, the input/output controller 284 is coupled to one or more motor controllers 290 and provides instructions and/or data to at least a subset of the one or more motor controllers 290, which, in turn, provide control signals to selected motors 292. In some embodiments, the one or more motor controllers 290 are integrated with the input/output controller 284 and the input/output controller 284 provides control signals directly to the one or more motors 292 (e.g., without a separate actuator controller).

The system 279 also includes a shunting component 294 (e.g., the shunting circuit 400 or 470). In some embodiments, the shunting component 294 is coupled to (e.g., in communication with) the input/output controller 284 and/or the motor controllers 290. In some embodiments, the shunting component 294 is coupled to (e.g., in communication with) one or more of the motors 292. For example, the shunting component 294 receives a fault signal from the input/output controller 284 or the motor controllers 290 and, in response to the fault signal, shunts a corresponding motor 292 (e.g., the motor 302). In some embodiments, the shunting component 294 is a hardware component (e.g., a circuit) that operates independently of software or firmware of the system 279. In some embodiments, the motor controllers 290 includes the shunting component 294.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code, or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database, or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

### 4. Illustration of Subject Technology as Clauses

Some embodiments or implementations are described with respect to the following clauses:

Clause 1. A medical system, comprising:
a motor of a robotic component;
a driver circuit coupled to the motor; and
a shunting circuit coupled to the driver circuit and configured to shunt the driver circuit for a set amount of time to stop the motor, the shunting circuit comprising a capacitor (C) and a first resistor (R), the capacitor and first resistor having an RC time constant, wherein the set amount of time corresponds to the RC time constant.

Clause 2. The medical system of Clause 1, wherein the shunting circuit comprises:
a first switch coupled between the driver circuit and a voltage source;
a second switch coupled between the driver circuit and an electrical ground; and
a relay component coupled to a plurality of fault signal inputs and configured to:
in response to a fault signal at one of the plurality of fault signal inputs, open the first switch and close the second switch; and
after the set amount of time from receiving the fault signal, close the first switch and open the second switch.

Clause 3. The medical system of Clause 2, wherein the relay component comprises a solid-state relay.

Clause 4. The medical system of Clause 2 or Clause 3, wherein the plurality of fault signal inputs includes at least one of:
an emergency stop signal;
a fault signal from the voltage source; or
a fault signal from the driver circuit.

Clause 5. The medical system of any of Clauses 2-4, wherein the shunting circuit comprises a second resistor coupled in series with the second switch.

Clause 6. The medical system of any of Clauses 1-5, wherein the driver circuit comprises a set of diodes in parallel with a set of phase transistors, and wherein the shunting circuit shunts the driver circuit by creating a path to electrical ground via the set of diodes.

Clause 7. The medical system of any of Clauses 1-6, wherein the robotic component comprises a robotic arm, and wherein the motor controls a joint of the robot arm.

Clause 8. The medical system of Clause 7, wherein the robotic arm is backdrivable after the set amount of time.

Clause 9. The medical system of any of Clauses 1-8, wherein the motor comprises a three-phase motor, and wherein the driver circuit comprises a set of transistors for each phase of the motor.

Clause 10. The medical system of any of Clauses 1-9, wherein the medical system is configured to test the shunting circuit as part of a start-up process.

Clause 11. A method for stopping a motor of a robotic component, comprising:
driving the motor via a driver circuit;
while the motor is being driven, receiving a fault signal;
in response to the fault signal, shunting the driver circuit to stop the motor, including:
decoupling the driver circuit for the motor from a voltage source; and
coupling the driver circuit to an electrical ground via a resistor, wherein the driver circuit is decoupled from the electrical ground in accordance with charge draining from a capacitor.

Clause 12. The method of Clause 11, wherein:
decoupling the driver circuit from the voltage source comprises opening a first switch coupled between the driver circuit and the voltage source; and
coupling the driver circuit to the electrical ground comprises closing a second switch coupled between the driver circuit and the electrical ground.

Clause 13. The method of Clause 12, wherein shunting the driver circuit comprises directing current from the motor to the resistor coupled in series with the second switch.

Clause 14. The method of any of Clauses 11-13, wherein the fault signal is one of:
an emergency stop signal;
a signal from the voltage source; or
a signal from the driver circuit.

Clause 15. The method of any of Clauses 11-14, wherein the driver circuit comprises a set of diodes in parallel with a set of phase transistors, and wherein shunting the driver circuit comprises creating a path to the electrical ground via the set of diodes.

Clause 16. The method of any of Clauses 11-15, wherein the motor controls a joint of the robotic component, and the method further comprises manipulating movement of the robotic component by adjusting operation of the motor.

Clause 17. The method of any of Clauses 11-16, further comprising, after decoupling the driver circuit from the electrical ground in accordance with the charge draining from the capacitor, maintaining the driver circuit in a backdrivable state, wherein in the backdrivable state the driver circuit is decoupled from the voltage source and decoupled from the electrical ground.

Clause 18, The method of any of Clauses 11-17, further comprising testing the shunting circuit as part of a start-up process for the robotic component.

Clause 19. A medical system, comprising:
a motor of a robotic component;
a driver circuit coupled to the motor;
a shunting circuit coupled to the driver circuit and configured to shunt the driver circuit to stop
the motor, the shunting circuit comprising:
a first switch coupling the driver circuit to a voltage source;
a second switch coupling the driver circuit to an electrical ground;
a current sensor coupled to the driver circuit and configured to measure a current in the driver circuit; and
a control component coupled to the current sensor, the first switch, and the second switch, the control component configured to, in response to a fault signal:
open the first switch; and
selectively open and close the second switch to maintain the current in the driver circuit within a set range while stopping the motor.

Clause 20. A method for stopping a motor of a robotic component, comprising:
driving the motor via a driver circuit;
while the motor is being driven, receiving a fault signal;
in response to the fault signal, shunting the driver circuit to stop the motor, including:
decoupling the driver circuit for the motor from a voltage source; and
selectively coupling the driver circuit to an electrical ground until the motor stops, including:
monitoring a current in the driver circuit;
in accordance with the current exceeding a first predetermined threshold, decoupling the driver circuit from the electrical ground; and
in accordance with the current going below a second predetermined threshold, recoupling the driver circuit to the electrical ground.

Clause 21. A medical system, comprising:
a motor of a robotic component;
a driver circuit coupled to the motor; and
a shunting circuit coupled to the driver circuit, the shunting circuit comprising:
a switch electrically coupled between a high voltage line of the driver circuit and an electrical ground;
a resistor electrically coupled between the high voltage line of the driver circuit and the electrical ground; and
a capacitor coupled to the switch for shunting the driver circuit over a set amount of time.

What is claimed is:

1. A medical system, comprising:
a motor of a robotic component;
a driver circuit coupled to the motor; and
a shunting circuit coupled to the driver circuit and configured to shunt the driver circuit for a set amount of time to stop the motor, the shunting circuit comprising a capacitor (C) and a first resistor (R), the capacitor and first resistor having an RC time constant, wherein the set amount of time corresponds to the RC time constant.

2. The medical system of claim 1, wherein the shunting circuit comprises:
a first switch coupled between the driver circuit and a voltage source;
a second switch coupled between the driver circuit and an electrical ground; and
a relay component coupled to a plurality of fault signal inputs and configured to:
in response to a fault signal at one of the plurality of fault signal inputs, open the first switch and close the second switch; and
after the set amount of time from receiving the fault signal, close the first switch and open the second switch.

3. The medical system of claim 2, wherein the relay component comprises a solid-state relay.

4. The medical system of claim 2, wherein the plurality of fault signal inputs includes at least one of:
an emergency stop signal;
a fault signal from the voltage source; or
a fault signal from the driver circuit.

5. The medical system of claim 2, wherein the shunting circuit comprises a second resistor coupled in series with the second switch.

6. The medical system of claim 1, wherein the driver circuit comprises a set of diodes in parallel with a set of phase transistors, and wherein the shunting circuit shunts the driver circuit by creating a path to electrical ground via the set of diodes.

7. The medical system of claim 1, wherein the robotic component comprises a robotic arm, and wherein the motor controls a joint of the robot arm.

8. The medical system of claim 7, wherein the robotic arm is backdrivable after the set amount of time.

9. The medical system of claim 1, wherein the motor comprises a three-phase motor, and wherein the driver circuit comprises a set of transistors for each phase of the motor.

10. The medical system of claim 1, wherein the medical system is configured to test the shunting circuit as part of a start-up process.

11. A method for stopping a motor of a robotic component, comprising:
driving the motor via a driver circuit;
while the motor is being driven, receiving a fault signal;
in response to the fault signal, shunting the driver circuit to stop the motor, including:
decoupling the driver circuit for the motor from a voltage source; and
coupling the driver circuit to an electrical ground via a resistor, wherein the driver circuit is decoupled from the electrical ground in accordance with charge draining from a capacitor.

12. The method of claim 11, wherein:
decoupling the driver circuit from the voltage source comprises opening a first switch coupled between the driver circuit and the voltage source; and
coupling the driver circuit to the electrical ground comprises closing a second switch coupled between the driver circuit and the electrical ground.

13. The method of claim 12, wherein shunting the driver circuit comprises directing current from the motor to the resistor coupled in series with the second switch.

14. The method of claim 11, wherein the fault signal is one of:
an emergency stop signal;
a signal from the voltage source; or
a signal from the driver circuit.

15. The method of claim 11, wherein the driver circuit comprises a set of diodes in parallel with a set of phase transistors, and wherein shunting the driver circuit comprises creating a path to the electrical ground via the set of diodes.

16. The method of claim 11, wherein the motor controls a joint of the robotic component, and the method further comprises manipulating movement of the robotic component by adjusting operation of the motor.

17. The method of claim 11, further comprising, after decoupling the driver circuit from the electrical ground in accordance with the charge draining from the capacitor, maintaining the driver circuit in a backdrivable state, wherein in the backdrivable state the driver circuit is decoupled from the voltage source and decoupled from the electrical ground.

18. The method of claim 11, further comprising testing the shunting circuit as part of a start-up process for the robotic component.

19. A medical system, comprising:
a motor of a robotic component;
a driver circuit coupled to the motor; and
a shunting circuit coupled to the driver circuit, the shunting circuit comprising:
a switch electrically coupled between a high voltage line of the driver circuit and an electrical ground;
a resistor electrically coupled between the high voltage line of the driver circuit and the electrical ground; and
a capacitor coupled to the switch for shunting the driver circuit over a set amount of time.
